(19) United States
(12) Patent Application Publication
(10) Pub. No.: US 2006/0051431 A1
(43) Pub. Date: Mar. 9, 2006

(54) Methods of managing multi-tissue injuries

(75) Inventor: Thomas Ray Becker, Libertyville, IL (US)

Correspondence Address: Jason J. Derry;McDonnell Boehnen Hulbert & Berghoff LLP, 31st Floor, 300 S. Wacker Drive, Chicago, IL, 60606, US

(73) Assignee: Regen-It, Inc., Gurnee, IL (US)

(21) Appl. No.: 11/141,963

(22) Filed: Jun. 1, 2005

Related U.S. Application Data
(60) Provisional application No. 60/577,948, filed on Jun. 8, 2004.
(60) Provisional application No. 60/578,787, filed on Jun. 10, 2004.
(60) Provisional application No. 60/580,088, filed on Jun. 16, 2004.
(60) Provisional application No. 60/593,012, filed on Jul. 30, 2004.

(51) Int. Cl. A61K 33/38; A61L 15/00

(52) U.S. Cl. 424/618; 424/445

(57) ABSTRACT

The invention provides methods of managing multi-tissue injuries under tissue regenerating conditions, the methods comprising contacting injured tissues with a silver ion based topical pharmaceutical composition and covering the injury with an occlusive dressing. Methods of the invention can restore flexibility, sensation, and appearance of damaged tissues.

This application is related to and claims priority to U.S. provisional application Ser. No. 60/577,948, filed Jun. 8, 2004; U.S. provisional application Ser. No. 60/578,787, filed Jun. 10, 2004; U.S. provisional application Ser. No. 60/580,088, filed Jun. 16, 2004; and U.S. provisional application Ser. No. 60/593,012, filed Jul. 30, 2004; the disclosure of each of which is incorporated by reference herein.

### FIELD OF THE INVENTION

The invention relates to managing multi-tissue injuries, particularly multi-tissue digit injuries, such as fingertip amputations. The methods of the invention create microenvironments, referred to herein as “tissue regenerating conditions” (TRC), that mimic pre-regeneration physiologic onsets and invoke genetically programmed tissue regeneration rather than scarification or tissue replacement. Thus, the methods of the invention prime the new wounds into TRC, which allows activation of various genetic signaling pathways necessary for total or near total recovery. The methods of the invention relate to contacting injured tissues with a silver ion based topical pharmaceutical composition and covering the injury with an air and water occlusive dressing, which is then protected by a hard covering. The hard covering is secured with standard known methods.

### BACKGROUND OF THE INVENTION

Fingertip amputations are very common injuries. Several incidents can cause such injuries, including, for example, accidents of saw blades, knives, meat cutters, turning gears, closing doors, snow blower, and lawn mower blades. Generally, treating fingertip amputations involves either skin grafting or scarification healing.

Scarification healing involves cells at the edges of an injury via collagen and elastic fiber extending into the wound to shrink the size of the defect and eventual scar coverage. These repairs do not restore severed nerves, blood vessels, fatty tissue or normal skin. A visible scar is produced by this healing process, which generally results in numbness and circulatory inadequacy in and around the region of the repaired tissues. Since nerve tissue is not restored neuromas are usually if not always formed at the end of the cut nerves.

Another type of healing of tissue injuries involves tissue replacement, which transplants cells within the same body from one area (donor site) into the wound. The growth rate of cells during tissue replacement increases in the injured area to produce enough cells to cover the injury. This method replaces the tissue lost with tissue composition similar to that of the donor site tissue. Replacement is only effective when proliferation of the homologous cells covers the entire injured area. Also, not all tissue injuries can be treated by the replacement healing method. For example, skin wounds with exposed bone must be converted to a boneless wound usually accomplished by cutting back the bone. This results in loss of length. These wounds are repaired with donor skin with limited epithelia and tissue restoration, poorly innervated, and abnormal inelastic skin. The subcutaneous soft tissue underneath the wound heals primarily by scarification.

In theory, tissue regeneration is superior to scarification and tissue replacement types of healing. Regeneration generally occurs in two stages. In the first stage, normal, mature cells at the site of the injury de-differentiate (i.e. revert to an embryonic, unspecialized state). The cells are then activated by various inducers to multiply rapidly, resulting in a mass of embryonic cells known as the blastema. The blastema is an essential element for regeneration of lost tissues, and must be an adequate size to allow complete regeneration to occur. A less than adequate size blastema will result in partial or incomplete regeneration, for example, an incomplete limb or regenerated individual tissue types that fail to organize into a complete structure. In the stage two of the regeneration process, the embryonic cells of the blastema differentiate into the specific cell types necessary to restore the missing tissues and organized structures in complete anatomical detail. The regeneration process is basically a recapitulation of the original embryonic development of the tissues being replaced.

Regenerative healing is common in certain species of amphibians and worms. In such amphibians, entire limbs can be regenerated, for example, in salamanders. In humans, the liver is known to undergo limited regeneration in response to injury, but limb and digit regeneration in adult humans is rare. In some cases, young children can have distal fingertip regeneration when the wound is not closed.

Currently, conventional methods of treating fingertip amputations primarily involve scarification healing or tissue replacement resulting in substantial or complete loss of flexibility sensation and resulting in neuroma formation. A method for genetically programmed regeneration of tissues in a human, particularly a human adult, is highly desirable. Such treatment methods would expedite patient outcome while restoring flexibility, function, and sensation; and, therefore, such methods would be more cost-effective than traditional treatment methods.

### SUMMARY OF THE INVENTION

The invention provides methods of managing multi-tissue injuries under conditions that induce regeneration of the damaged tissues, including dermis, fat, neural, and vascular tissues. Such conditions are referred to herein as “tissue regenerating conditions” (TRC). The methods of the invention can restore sensation, appearance, and flexibility to a multi-tissue injury, particularly a digit injury, thereby eliminating or reducing disability typically associated with such injuries.

In one aspect, the methods of the invention comprise contacting a multi-tissue injury with a silver ion based topical pharmaceutical composition, such as a silver sulfadiazine cream, under tissue regenerating conditions. In a further aspect, the injury is covered with an occlusive dressing after the injury is contacted with a silver ion based cream, thereby preventing exposure of the injury to air and water, which is detrimental to the formation of the precursor of the would be regenerated tissues. The precursor is a clear raw egg white like substance formed at the early stages layered and extended from the wounds. In another aspect, the management methods of the invention comprise avoiding contact of the injury with anything other than a silver ion based topical pharmaceutical composition after the injury is initially cleaned, and debrided. In yet another aspect, a protective cover is used to cover the injury and surrounding normal tissues, which allows the precursor of the regenerating tissues to develop maximally.

Traditional methods of treating multi-tissue injuries have used silver ion based creams to protect the injury from infections. While the methods of the invention comprise contacting an injury with a silver ion based topical pharmaceutical composition, such as silver ion based creams, the invention provides an improvement on such traditional methods, wherein the improvement comprises preventing exposure of the injury to air and water that may be detrimental to the precursor of the would be regenerated tissues.

In addition, the invention provides kits for managing multi-tissue injuries comprising but not limited to: silver ion based topical pharmaceutical composition; an occlusive dressing, a standard dressing; and/or a protective cover.

Specific preferred embodiments of the invention will become evident from the following more detailed description of certain preferred embodiments and the claims.

### DETAILED DESCRIPTION OF THE DRAWINGS

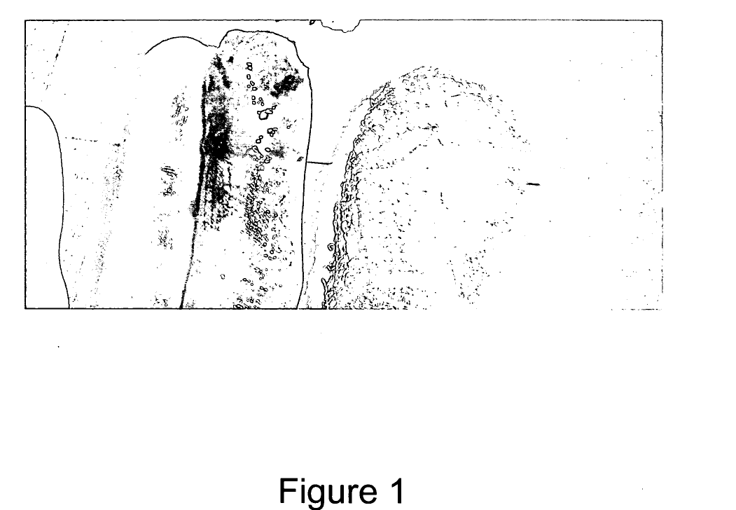

FIG. 1 shows a fingertip amputation of a 42-year old male patient taken 2 days after injury.

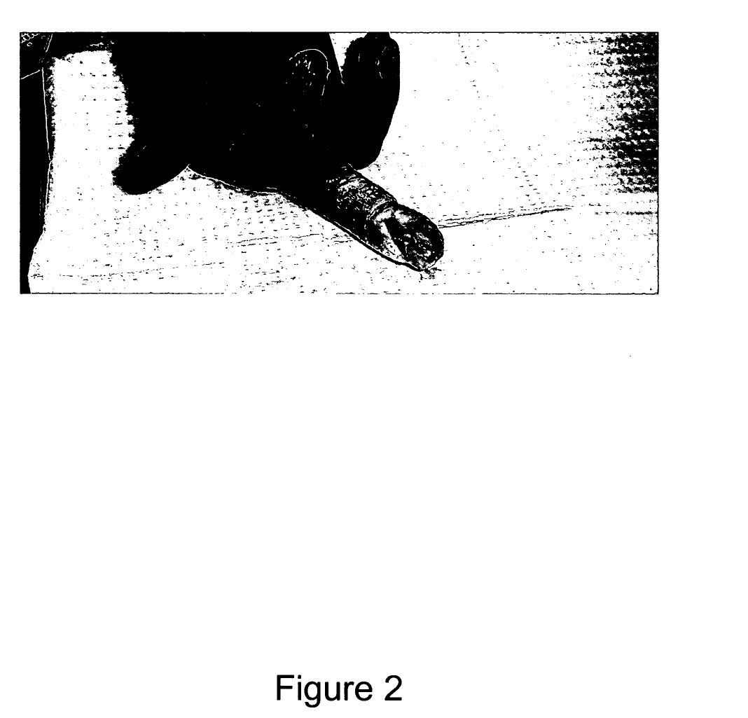

FIG. 2 shows the injured finger at 4 days after treatment (Phase 2).

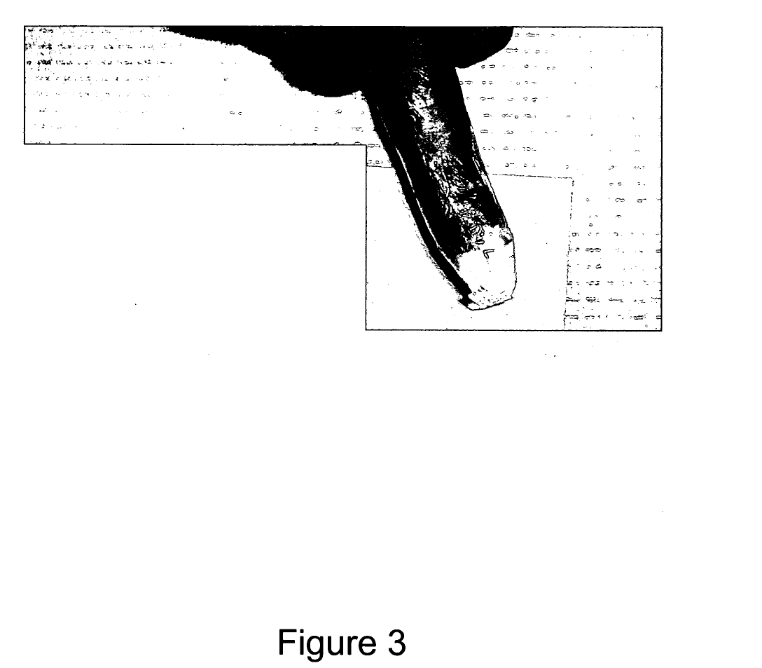

FIG. 3 shows the injured finger at 7 weeks after treatment with the occlusive dressing still on the finger (Phase 3).

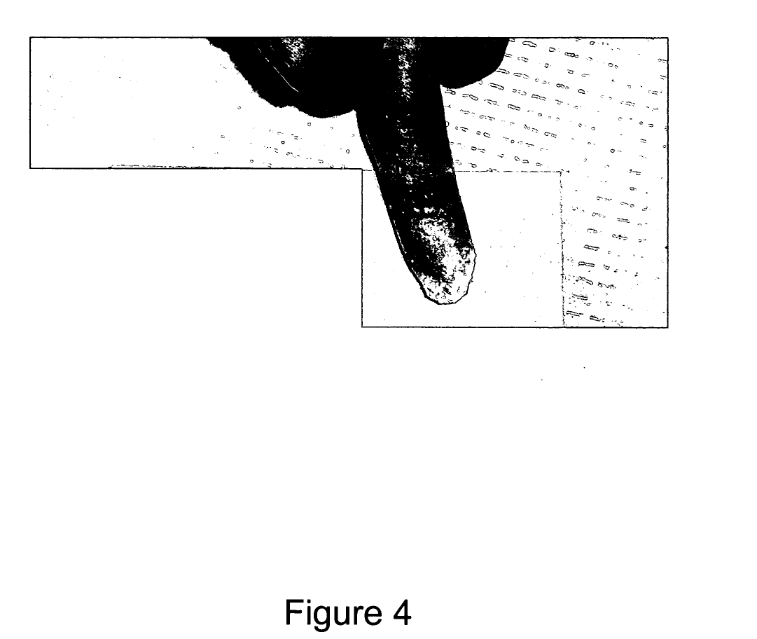

FIG. 4 shows the injured finger at 7 weeks after treatment without the occlusive dressing (Phase 3).

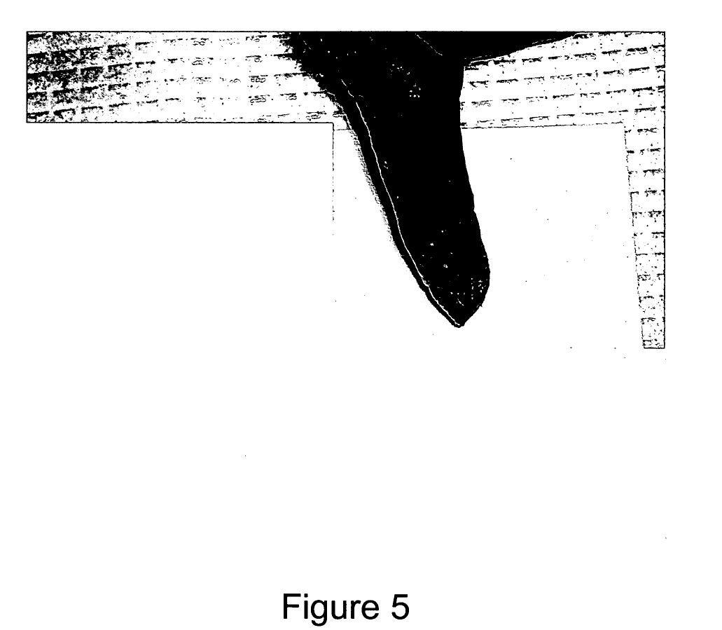

FIG. 5 shows the injured finger at 9 weeks after treatment (Phase 4).

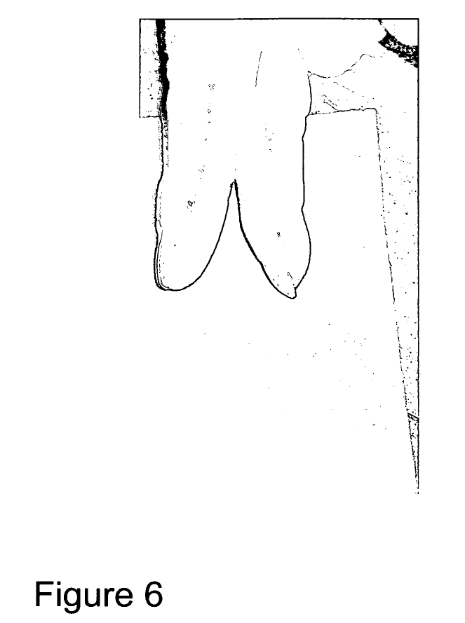

FIG. 6 shows the injured finger upon completion of treatment.

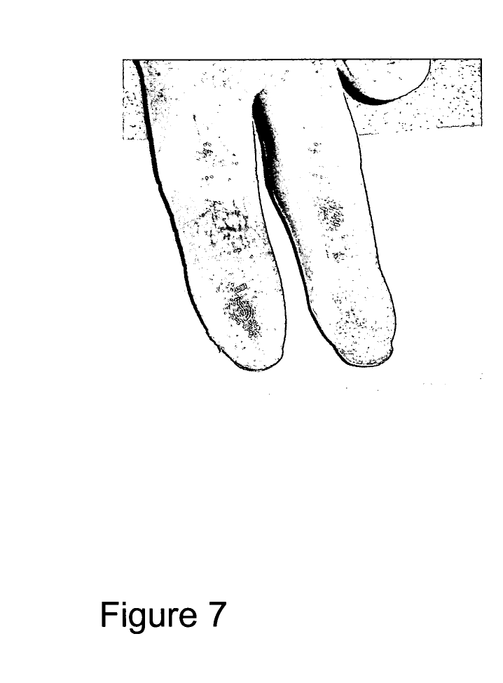

FIG. 7 shows an example of completion of treatment demonstrated by reformation of fingerprint lines.

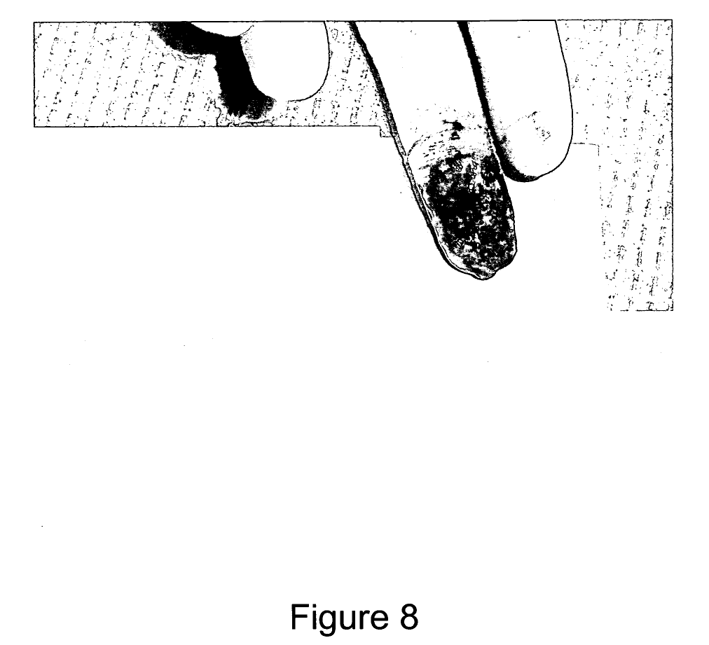

FIG. 8 shows an example of precursor of would be regenerated tissues, which typically appear as a clear raw egg white substance layered and extended from the wound.

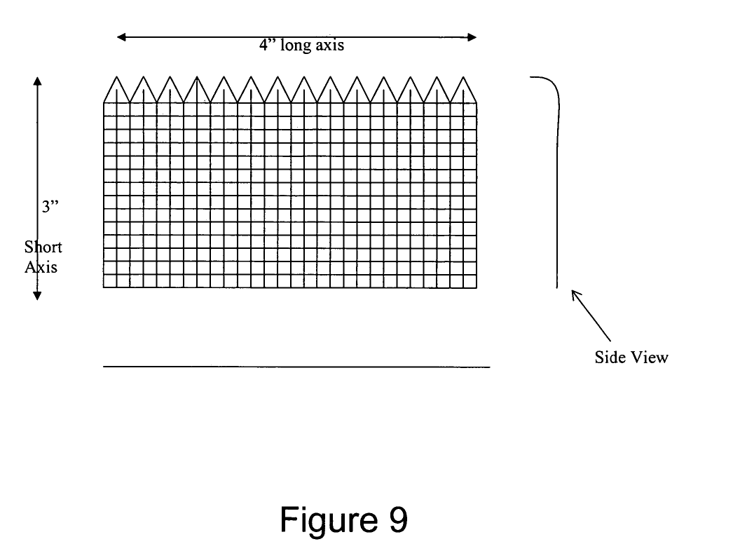

FIG. 9 shows an example of a protective cover of the invention.

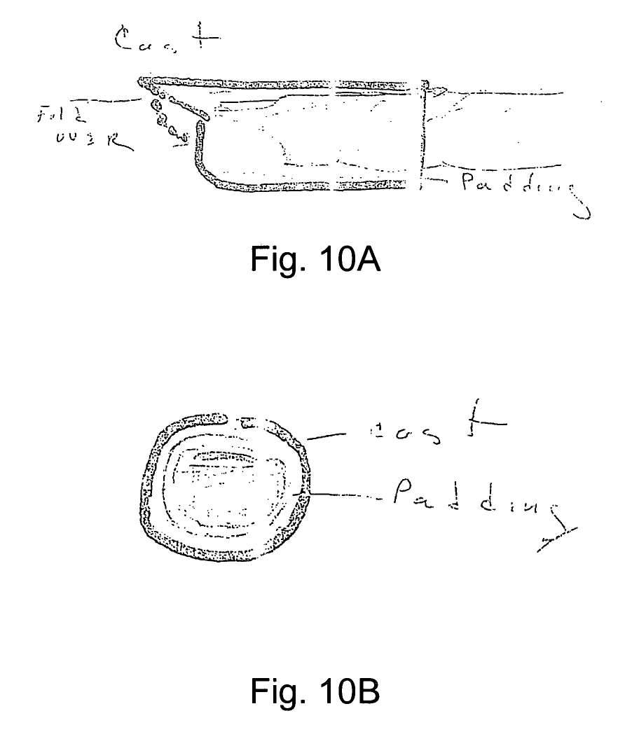

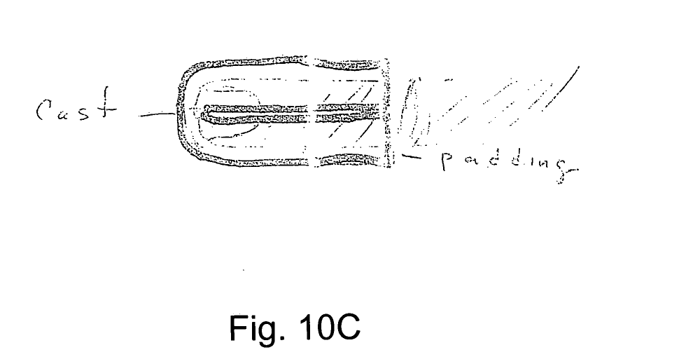

FIGS. 10A, 10B, and 10C show various views of an example of a splint of the invention.

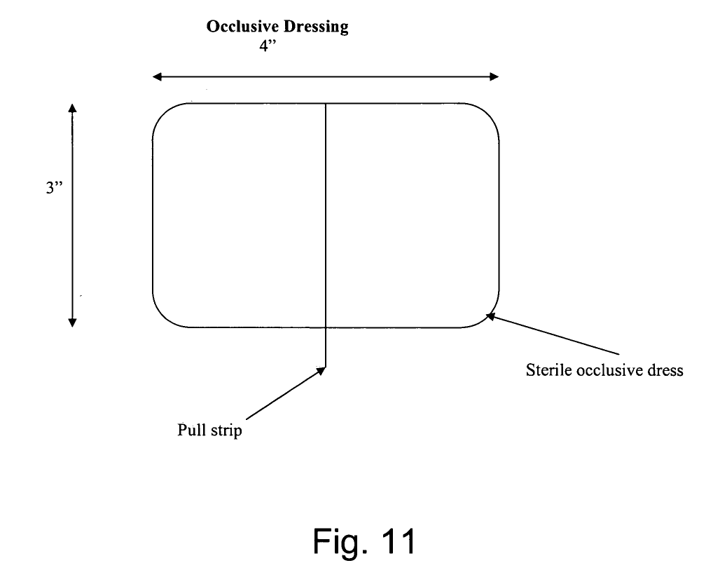

FIG. 11 shows an example of an occlusive dressing of the invention.

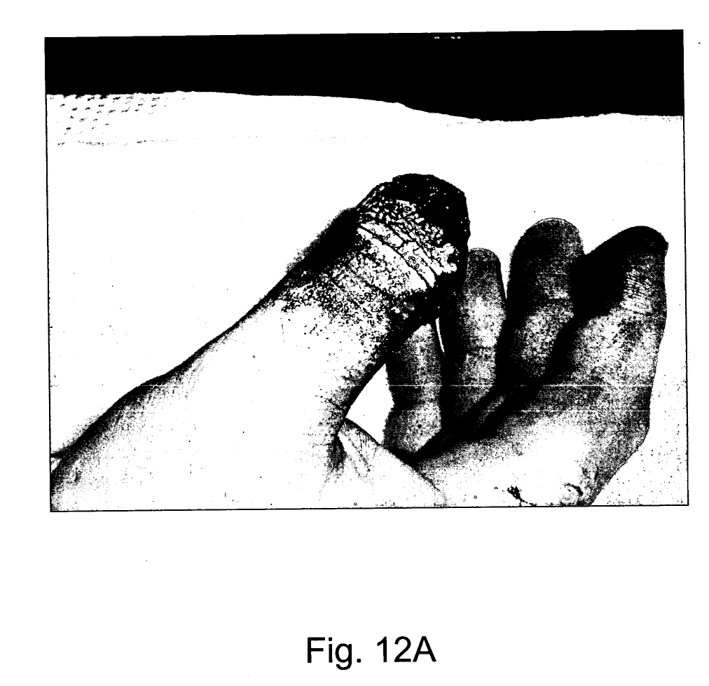

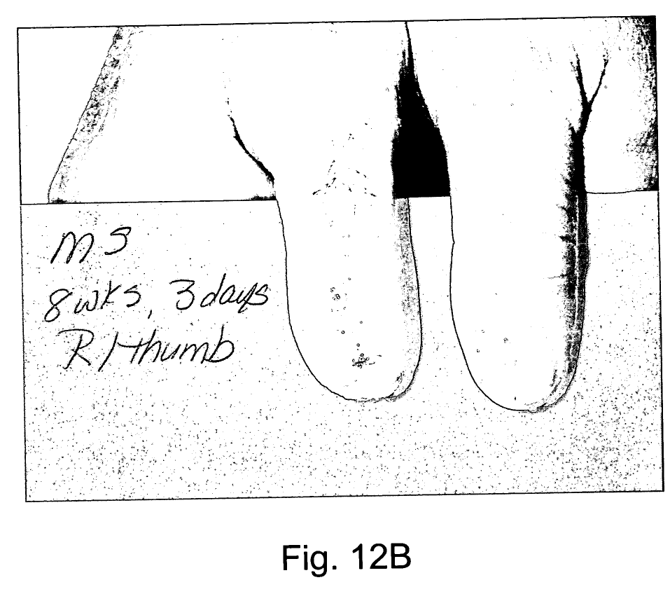

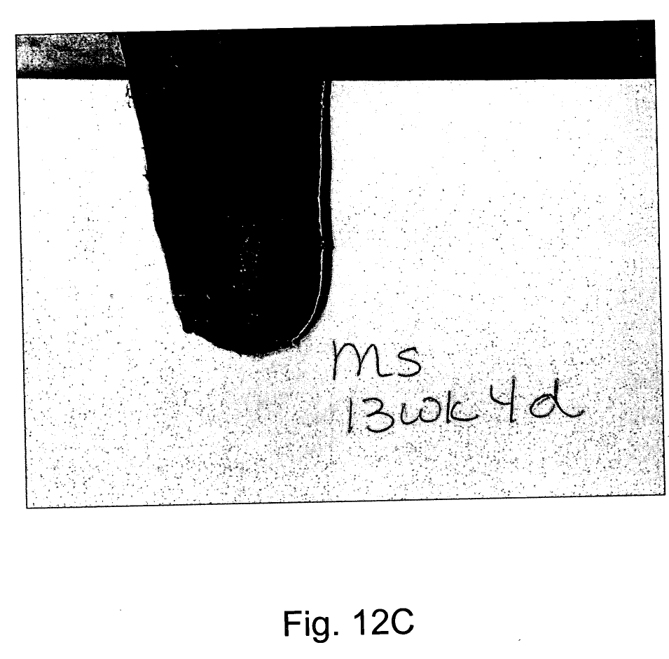

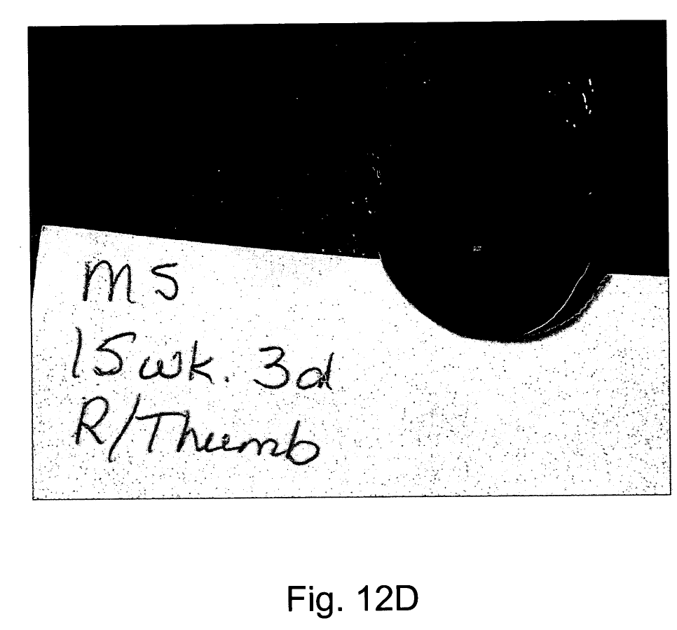

FIG. 12A shows a thumb with a Type 3 injury shown 3 days after injury.

FIG. 12B shows the injured thumb (left) next to the uninjured thumb (right) after 8 weeks and 3 days of treatment with the method of the invention.

FIG. 12C shows the thumb after 13 weeks and 4 days of treatment with the method of the invention.

FIG. 12D shows the thumb after 15 weeks and 3 days of treatment with the method of the invention.

### DETAILED DESCRIPTION OF THE PREFERRED EMBODIMENTS

The methods of the invention can be used to induce regeneration of damaged dermal tissues and associated vascular, neural, and other tissues, including formation of the fingerprints. Reformation of the fingerprint, as shown in FIG. 7, demonstrates that the methods of the invention stimulate a genetically programmed process that formed the original fingerprint. In one embodiment, the damaged tissues are associated with digits (i.e. fingers or toes), for example, when multiple tissue layers on the tip of the finger are amputated or cut away from the digit. Typically, such injuries are treated by cleaning the surrounding skin and injured area with a bacteriasidal agent, surgically removing any devitalized tissues, trimming back exposed bone (if any), and covering the injury with a standard dressing until the area has scarred over, or by one of several skin graft methods. The invention provides methods for regenerating tissues over an injury rather than allowing scarification or requiring skin grafting techniques with their donor site morbidity.

In one embodiment, the invention provides methods of treating a multi-tissue injury, preferably a multi-tissue digit injury, wherein bone is not exposed by the injury. In this embodiment, a method of the invention comprises the steps of covering the multi-tissue injury and adjacent normal skin with an occlusive dressing and covering the occlusive dressing with a protective cover. A standard dressing is placed over the occlusive dressing before it is covered with the protective cover. The silver ion based topical pharmaceutical composition, such as a silver sulfadiazine cream, will be absorbed into the injured area after a certain amount of exposure. Thus, in another embodiment, the protective cover and occlusive dressing are removed, without disturbing the injury, and the injury is contacted with additional amounts of the silver ion based topical pharmaceutical composition. The adjacent normal skin can be cleaned during this step.

Afterward, the injury and adjacent normal skin are again covered by an occlusive dressing and a protective cover. The protective cover can be secured, for example, by 3M Coban™ wrap (3M, St. Paul, Minn.) or any such standard means known in the art, to the area. In certain embodiments, these steps are repeated every 1, 2, 3, or 4 days (during phase 1) until formation of the precursor of would be regenerated tissues. The precursor typically appears as a clear raw egg white substance layered and extended from the wound (see FIG. 8). Preferably, the steps are repeated every 4 days (phase 2). The steps are repeated weekly (phase 3) until treatment is complete (phase 4). Phase 4 is the final period where skin matures; a crust may appear that loosens with time or the normal looking new tissue turns black from the silver ion cream. The silver ion based pharmaceutical composition is applied daily to keep the area moist without an occlusive dressing, but the skin is protected from injury. Treatment is considered complete when neural, vascular, dermal, and other tissues have re-grown over the injury and the regenerated tissue resembles normal, healthy tissue in appearance and sensation. In a particular embodiment, a completed treatment is hallmarked by complete skin cover and the appearance of fingerprint lines, or a hard crust of dead epithelium that loosens, and blackening of the area due to the silver ion based topical pharmaceutical composition.

In another embodiment, the invention provides methods of treating a multi-tissue injury, preferably a multi-tissue digit injury, wherein bone is exposed by the injury. In injuries where bone is exposed, a large amount of fluid is produced by the interaction of the wound with the silver ion based topical pharmaceutical composition under tissue regenerating conditions (TRC). The tissue regenerating conditions for an injury where bone is exposed comprise steps that permit drainage of body fluids from the injured area. Thus, in certain embodiments, a method of the invention comprises the steps of contacting the injury with a silver ion based topical pharmaceutical composition, such as a silver sulfadiazine cream, wherein normal skin adjacent to the injury is not contacted with the silver ion based topical pharmaceutical composition; covering the injury with a standard dressing and/or perforated occlusive dressing; and covering the dressing(s) with a protective cover. The dressing(s) and protective cover are then removed and the adjacent normal skin is cleaned and dried while the injury is contacted with additional silver ion based topical pharmaceutical composition, as discussed above. These steps are repeated until drainage from the injury has stopped. In certain embodiments, these steps are repeated every 1, 2, 3 or 4 days until drainage stops.

Once drainage has stopped, the injury and adjacent normal skin are covered with an occlusive dressing and the occlusive dressing is covered with a protective cover. The protective cover can be secured to the injured part, for example, by 3M Coban™ wrap (3M, St. Paul, Minn.) or any standard securing means known in the art. A standard dressing is placed over the occlusive dressing before covering with the protective cover. The silver ion based topical pharmaceutical composition will be absorbed into the injured area after a certain amount of exposure. Thus, in another embodiment, the protective cover and occlusive dressing are removed, without disturbing the injury, and the injury is contacted with additional amounts of silver ion based topical pharmaceutical composition. The adjacent normal skin can be cleaned during this step. Afterward, the injury is covered with the silver ion topical pharmaceutical composition, and the injury and adjacent normal skin are covered by an occlusive dressing, padding, and a protective cover. The protective cover can be secured to the extremity, for example, with 3M Coban™ wrap (3M, St. Paul, Minn.) or other standard methods. In certain embodiments, these steps are repeated every 3 or 4 days after formation of the precursor of would be regenerated tissues under tissue regenerating conditions. The precursor is a clear raw egg white substance layered and extended from the wound initiating (phase 2). Preferably, the steps are repeated every 3 or 4 days in phase 2 until the tissue is restored with dermal coverage (phase 3). Then, the steps are repeated weekly until skin cover is complete, initiating (phase 4). As discussed above, treatment is considered complete when neural, vascular, dermal, and other tissues have re-grown over the injury, and the regenerated tissue resembles normal, healthy tissue in appearance and sensation. In a particular embodiment, a completed treatment is hallmarked by complete skin cover and the appearance of fingerprint like lines, or a hard crust of dead epithelium that loosens, and/or blackening of the area due to the silver ion based topical pharmaceutical composition. These conditions hallmark phase 4 where the occlusive dressing is stopped, silver ion based topical pharmaceutical composition is applied daily without an occlusive dressing, and a protective cover is worn for activities until skin sensitivity is diminished.

In certain other embodiments, removal of any dressing and/or cover during a method of the invention is performed in such a way that the injury is not further damaged. For example, dressings are not pulled off, but are carefully removed to avoid further trauma. Disturbing the wound during dressing or cover removal can cause scar tissue to develop.

In certain embodiments, routine cleaning comprises saline flushing and debriding performed at the initial visit only. The injury is not touched at anytime thereafter until the treatment is complete. Thus, the injury is left as an open wound throughout treatment, and the open wound is not touched, cleaned, or dried after initial treatment. Touching the open wound can initiate scar tissue formation, which can interfere with re-growth of the appropriate tissues.

In another embodiment, the methods of the invention can be used to replace scar tissue with regenerated tissues. In this embodiment, the methods outlined herein are preceded by surgical removal of the scar tissue (i.e. the multi-tissue injury is intentionally created surgically). In other embodiments, the methods of the invention can be used to surgically create a fresh amputation from an old healed injury to allow regeneration, thus restoring sensation to sensory deprived areas. In addition, digital neuromas can be treated using methods of the invention by cutting out the neuroma and surrounding tissue to stimulate regeneration of normal terminal nerve endings. In a particular embodiment, one of skill in the art will recognize that these methods of the invention are not limited to digit injuries, but can be applied to regeneration of any dermal and associated tissues.

In yet another embodiment, the invention provides a kit for managing multi-tissue injuries comprising: silver ion based topical pharmaceutical composition, such as a silver sulfadiazine cream, a standard dressing, a protective cover, and/or an occlusive dressing, each in a quantity sufficient for treating a multi-tissue injury for at least one treatment. In addition, a kit of the invention can comprise instructions for managing a multi-tissue injury. The kit can comprise additional components, for example, ointments, sprays, or creams that are useful for cleaning an injury and/or protecting an injury from infection.

In certain embodiments, the methods described herein can be used to treat Type 1, Type 2, and Type 3 injuries. A Type 1 injury is a soft tissue amputation, traditionally treated with skin grafts and/or granulating covers. A Type 2 injury is a soft tissue plus bone through a portion of the nail (e.g. fingernail), traditionally treated by shortening the bone and skin grafts, skin flaps, or a “VY” closure (see, for example, Jackson, 2001, American Family Physician 64:455-458). A Type 3 injury is a soft tissue plus bone proximal to the nail, typically treated by disarticulation of the end of the finger. The time frame for regeneration stimulated by the methods of the invention on a Type 1, Type 2, or Type 3 injury is generally about 3 months. The methods of the invention can restore finger length (i.e. greater than or equal to finger length at the time of injury), sensation, and cause no neuromas and good cosmesis. The example herein describes the methods of the invention as applied to a Type 2 injury (results shown in FIGS. 1-7. FIGS. 12A-D show the results of the methods of the invention applied to a Type 3 injury.

Definitions

As used herein, the phrase “tissue regenerating conditions” (TRC) refers to conditions described herein that promote genetically programmed regeneration of tissues rather than scarring. Specifically, tissue regenerating conditions comprise contacting a multi-tissue injury with a silver ion based topical pharmaceutical composition, covering the injury with an occlusive dressing to substantially prevent exposure of the injured tissues to air and/or water, and not traumatizing the injured tissues after an initial cleaning and/or debriding. Thus, in one embodiment of the invention, the injured tissues are only contacted with silver ion based topical pharmaceutical composition after initial cleaning and/or debriding, and are not disturbed otherwise.

As used herein, “Phase 1” refers to the period of treatment where interaction between the silver ion based pharmaceutical composition and body tissue fluids result in liquefaction. This phase starts from the time of the initial dressing until liquefaction stops and evidence of formation of a raw egg white appearance of the surface of the injured area is seen. The dressing is changed every 1, 2, 3, or 4 days during this phase. Typically the dressing is an occlusive dressing and/or a standard dressing. A hard cover is not essential during this phase, but may be beneficial in reducing the pain. Where bone is exposed by the injury, the occlusive dressing is preferably perforated during Phase 1 to facilitate drainage.

As used herein, “Phase 2” refers to the period of regeneration, from the formation of a raw egg white appearance of the surface of the injured area to the complete regeneration of the tissues. Complete covering implies new raw dermis (early skin cover) and thickening (i.e. drying out) of the topical pharmaceutical composition. The dressing (occlusive cover, padding or gauze, and hard cover) is changed every 3 or 4 days during this phase.

As used herein, “Phase 3” refers to the period of regeneration, from completion of dermal cover and thickening of the topical pharmaceutical composition to the formation of epidermis, including the appearance of normal tissue characteristics of the skin, a hard crust layer or blackening of the skin. The dressing (occlusive cover, padding or gauze, and hard cover) is changed weekly during this stage.

As used herein, “Phase 4” refers to the final period where skin matures; a crust may appear that loosens with time or the normal looking new tissue turns black from the silver ion based pharmaceutical composition. The silver ion based pharmaceutical composition is applied daily, to keep the new skin moist, without an occlusive dressing, but the skin is protected from injury with a protective cover. For heavy activity, a hard cover is worn. Water is avoided, but does not seem to be detrimental at the end of phase 4 when the process is complete. The skin at the end of Phase 4 is very sensitive and similar to the skin of a human baby. This new skin toughens up generally within several days.

Treatment is complete when the injured tissues have substantially or completely regenerated, such that the regenerated tissues have sensation and other features characteristic of normal, healthy tissues. For example, where a fingertip has regenerated, the tissues have sensation and fingerprint lines similar or identical to that of the pre-injured finger.

As used herein a “protective cover” refers to a device used to protect digits or appendages having multi-tissue injuries. A protective cover can be applied, for example, over a digit to allow regeneration of tissues without the tissues being further damaged by everyday activities or other trauma. When the injury is a digit injury, a protective cover can be the same size as the opposite digit (i.e. the same digit on the opposite hand or foot) or can extend beyond the injury to a length that equals the opposite digit (i.e. the digit plus the cover equals the length of the same digit on the opposite hand or foot). The protective cover allows re-growth to occur while preventing any contact with the injured area underneath. Non-limiting examples of suitable protective covers include a splint or a cast. For example, the methods described in the Examples herein include a fiberglass finger cast. Such a cast can be generated from a thin fiberglass sheet and shaped to conform to the injured digit.

In one embodiment, the invention provides a flexible, prefabricated protective digit cover, such as a splint or cast for a finger that can protect the finger from injury. In one aspect, the protective cover is a prefabricated finger cast that allows protection and maximizes function for a finger. In particular aspects, the protective cover of the invention comprises a material that: is semi-rigid in its short axis; is flexible in its long axis; porous to allow passage of fluids (e.g. body fluids or pharmaceutical liquids); has a rough inner surface to prevent slippage; and comprises a serrated edge along a short axis that folds at an angle (such as 90°) to the surface of the cover or is curved as shown in FIG. 9 (side view), so that when the cover is wrapped around a digit the serrated edges create an enclosure over the tip of the digit.

FIG. 9 shows an example of a protective cover of the invention. The cover can be applied to a digit by wrapping the cover around the digit. As shown in FIG. 9, one side of the long axis can be serrated. The serrated edge can also be curved, as shown by the side view in FIG. 9. The side view shows the cover as it lies on the outer surface with the serrated edge curved upward. When the cover is wrapped around a digit, the serrated edge encloses the tip of the digit to allow protection of the end of the digit. The dimensions of the cover shown in FIG. 9 are about 4″ on the long axis and 3″ on the short axis. In one embodiment, the cover can be cut along a non-serrated edge to provide a more appropriate length and/or width for a specific digit that needs to be protected. The material can comprise an inert, non-absorbent composite, such as fiberglass.

In another embodiment, the splint has a length of about 3 to about 4 inches; has a width of about 3 inches; has a serrated edge; and is made of a flexible material, such as fiberglass casting tape, which hardens when wet. The serrated edge can be about a ¼ to about ½ inch deep. Non-limiting examples of fiberglass casting tape include Caraglass (Carapace, Inc., New Tazewell, Tenn.) and Scotchcast™ (3M™, St. Paul, Minn.).

A splint of the invention can be packaged to avoid moisture, which can cause a material such as fiberglass casting tape to harden before application. For example, the splint can be packaged in an airtight, aluminum bag. Upon removing the splint from the packaging, the splint can be measured and cut to a desired size. The splint can then be wetted and applied to the finger over the dressing. The splint is applied with the serrated edge extending past the end of the finger (FIG. 10A). The splint is then wrapped with a self-adherent wrap, such as Coban™ (3M™, St. Paul, Minn.), to allow the splint to conform to the finger (see FIG. 10B). The serrated edge can be molded over of the digit to form a closure over the digit as the splint hardens and the self-adherent wrap is applied over the serrated edge. The serrated edge overlaps onto itself, while the linear edge does not overlap onto itself (i.e. when wrapped around a digit, one edge does not touch or barely touches the other edge to allow for swelling of the tissue and ease of removal) (FIG. 10C).

A “standard dressing” as used herein refers to a typically used wound dressing. Such dressings are known in the art. Non-limiting examples of such a “standard dressing” include gauze, foam padding, Silipos® layer, and cast padding. During a method of the invention, an injury is not subjected to further trauma (i.e. after initial cleaning and/or debriding) and is not exposed to air or water. Thus, any dressing or cover that can accommodate these conditions can be used in a method of the invention.

As used herein, an “occlusive dressing” is a bandage or dressing that closes a wound or damaged area of the body and prevents it from being exposed to the air and water and helps provide a tissue regenerating condition (TRC) environment. Non-limiting examples of such dressings include Tegaderm™ (3M, St. Paul, Minn.) and OpSite® (Smith and Nephew, United Kingdom). An occlusive dressing can also be perforated to facilitate drainage from an injured area. Generally, occlusive dressings are removed from a patient by cutting, peeling, or tearing the dressing away from the wound, which can further damage the wound underneath.

In one embodiment, the invention provides an occlusive dressing that can be easily removed, thus minimizing or completely avoiding traumatizing a wound underneath the dressing. Specifically, an occlusive dressing of the invention comprises an inner surface that can contact a wound when applied to a patient, an outer surface, and a seam running across the dressing as shown in the attached FIG. 11. In one aspect, the seam is perpendicular to the long axis of the dressing, as shown. In another aspect, the seam comprises a removing mechanism, such as a pull strip, that separates the dressing into two parts upon removal from the seam. For example, the pull strip can be a thread incorporated into the dressing that separates the dressing into two parts when pulled away from the dressing. In another embodiment, the edges of the seam are perforated and can be removed from the dressing by pulling the seam from one edge of the long axis to the opposite edge of the long axis of the dressing, thus separating the dressing into two parts for easy removal from the patient.

FIG. 11 provides an example of a removable occlusive dressing of the invention. The dressing shown in FIG. 1 shows a seam running across the dressing with a pull strip that can be pulled from one edge of the long axis of the dressing toward the opposite edge to separate the dressing into two parts. An occlusive dressing of the invention is not limited to what is shown in FIG. 11.

As used herein a “silver ion based topical pharmaceutical composition” is a pharmaceutical composition that comprises silver ion, and is especially adapted for topical administration. The term “pharmaceutical composition” as used herein refers to a composition capable of inducing a desired therapeutic effect when properly administered to a patient, together with pharmaceutically acceptable formula or carriers. The exipients or carriers suitable for the pharmaceutical composition of the invention are for routine formulations in cream, gel, paste, ointment, frost, powder, emulsion, or aerosol form.

In addition, pharmaceutical compositions of the invention can also comprise other traditional agents helpful in wound healing, such as anti-inflammatory agents, astringents, emollients or analgesics, provided that they are not detrimental to the expected result, that is, regeneration of tissues without scarification. Those ordinary persons skilled in the art know the incorporation of these traditional agents into the pharmaceutical compositions.

Non-limiting examples of silver ion based topical pharmaceutical compositions include sulfadiazine creams, such as Silvadene. The amount of a silver ion based cream used in a method of the invention will depend on various factors specific to the injury type. The quantity used is dependant on the size of the injury and the extent of the injury, but is at least sufficient to cover the injury with about ⅛″ thickness of the silver ion pharmaceutical composition.

In one embodiment, an occlusive dressing can comprise a silver ion based pharmaceutical composition either layered on a surface of the dressing or impregnated in the dressing.

As used herein, “physical trauma” refers to any contact of the injured tissues other than by a silver ion based topical pharmaceutical composition. For example, physical trauma could occur by touching the injured tissues with a swab or other cleaning device, allowing the tissues to dry out washing with water. The simple act of applying water to the TRC results in cell lyses. Physical trauma to injured tissue during regeneration can promote scarring and interrupt the regeneration process. Thus, removal of an occlusive dressing and protective cover must be carefully executed so as not to disturb the injury. Preferably, therefore, a dressing and/or cover are removed, not by pulling, but by unwrapping or opening in a manner that does not manipulate the injured tissues.

A “multi-tissue injury” is an injury that involves damage or loss of multiple tissues, for example, dermal, fat, neural, and/or vascular tissues. A multi-tissue injury can be caused by a variety of factors, including, for example, cuts, incisions (including surgical incisions), abrasions, lacerations, fractures, contusions, burns, and amputations. A multi-tissue injury can occur on the end of a digit (i.e. a partial amputation) or on a part of the body where the injury creates an open wound, for example, on a surface of the skin on an arm, leg, back, chest, or abdominal area.

The term “patient” as used herein includes human and animal subjects.

Unless otherwise required by context, singular terms shall include pluralities and plural terms shall include the singular.

### EXAMPLE

The following example, including the experiments conducted and results achieved are provided for illustrative purposes only and are not to be construed as limiting the invention.

A 42-year-old patient, a self-employed carpenter, presented with a crushed long and index finger of his hand, with amputation of the end of the long finger and bone. He was treated in the emergency room as follows: the wound was gently cleaned and soaked for 60 seconds in peroxide followed by betadine solution for an additional 60 seconds. The wound was covered with generic silver sulfadiazine cream 1%, an occlusive dressing, Tegaderm™ (3M, St. Paul, Minn.) and then roller gauze.

He followed up in the office two days later (FIG. 1). The dressing was removed. The surrounding normal skin was cleaned and dried. A ⅛″ layer of Silvadene (Monarch Pharmaceuticals, Inc., Bristol, Tenn.) was applied to the open wound. This was followed by a Tegaderm™ (3M, St. Paul, Minn.) sterile occlusive dressing, roller gauze, and a finger cast was made from fiberglas to the level of the PIP joint, and secured with a 3M Coban™ wrap (3M, St. Paul, Minn.). He was allowed to resume normal activities.

He followed up 4 days later for dressing change. After removal of the cast and padding, the occlusive dressing was gently cut and removed to avoid injury to the open wound. A white discoloration of the surrounding skin was observed caused by the continuous contact with fluid from the liquefaction stage (Phase 1) (FIG. 2). The surrounding normal skin was cleaned and dried. A ⅛″ layer of Silvadene (Monarch Pharmaceuticals, Inc., Bristol, Tenn.) was applied to the open wound and a Tegaderm™ (3M, St. Paul, Minn.) sterile occlusive dressing reapplied followed by 2″ roller gauze padding, and a fiberglass cast. The cast was secured to the wrist with 3M Coban™ wrap (3M, St. Paul, Minn.). This process was repeated every 3 to 4 days until 7+ weeks (Phase 3) (FIGS. 3 and 4).

He was then instructed in weekly dressing changes and seen in the office every two weeks. By 10 weeks he had reached early Phase 4 (FIG. 5). Part of the skin contained a black crust, which was hard, but not loose (i.e. firmly attached to underlying tissues). Protection was continued until the black crust came off. Finger sensitivity was treated with a protective silipose cover for activities only for approximately 4 weeks. FIG. 6 shows the healed finger after treatment was complete.

FIGS. 12A-12D show the results of the above described technique used to regenerate a severed thumb.

It should be understood that the foregoing disclosure emphasizes certain specific embodiments of the invention and that all modifications or alternatives equivalent thereto are within the spirit and scope of the invention as set forth in the appended claims.

What is claimed is:

1. A method of managing a multi-tissue injury comprising the steps of contacting the injury with a silver ion based topical pharmaceutical composition under tissue regenerating conditions (TRC) that avoids the exposure of air and water for a time that permits regeneration of damaged tissues.

2. The method of claim 1, wherein normal skin adjacent to the injury is not contacted with the silver ion based topical pharmaceutical composition.

3. The method of claim 1, wherein the tissue regenerating conditions comprise covering the injury and adjacent normal skin with an occlusive dressing, covering the dressing with a protective cover, and securing the protective cover over the injury.

4. The method of claim 3 comprising the steps of:
a. removing the protective cover and occlusive dressing;
b. cleaning and drying the adjacent normal skin;
c. contacting the injury with a silver ion based topical pharmaceutical composition, wherein normal skin adjacent to the injury is not contacted with the silver ion based topical pharmaceutical composition;
d. re-covering the injury and adjacent normal skin with an occlusive dressing and protective cover; and
e. securing the protective cover over the injury;
wherein the injury does not involve bone exposure.

5. The method of claim 4, wherein steps (a) through (e) are repeated until treatment is complete, wherein treatment is complete when neural, vascular, fat, and dermal tissues have re-grown over the injury and the skin shows visible finger print type lines.

6. The method of claim 4, wherein steps (a) through (e) are repeated every 1, 2, 3, or 4 days until the formation of the precursor of would be regenerated tissues.

7. The method of claim 6, wherein after the precursor has formed, steps (a) through (e) are repeated every 3 or 4 days until the injury is covered with dermis.

8. The method of claim 7, wherein after dermis has covered the injury, steps (a) through (e) are repeated weekly until epidermis is formed over the dermis.

9. The method of claim 8, wherein after epidermis is formed, the epidermis is contacted with a silver ion based topical pharmaceutical composition and covered with a protective cover, which are changed at least one time daily until treatment is complete.

10. The method of claim 1, wherein the injury is cleaned and debrided before contact with the silver ion based topical pharmaceutical composition.

11. The method of claim 10, wherein the injury is not touched, cleaned, or dried after it is cleaned and debrided.

12. The method of claim 1, wherein the injury is a digit injury.

13. The method of claim 1, wherein the silver ion based topical pharmaceutical composition is a silver sulfadiazine topical pharmaceutical composition.

14. The method of claim 1 comprising the steps of:
a. contacting the injury with silver ion topical pharmaceutical composition, wherein normal skin adjacent to the injury is not contacted with the silver ion based topical pharmaceutical composition;
b. covering the injury with a standard dressing or perforated occlusive dressing before the standard dressing;
c. covering the standard dressing with a protective cover;
d. securing the protective cover by standard methods;
e. removing the dressing and cover;
f. cleaning and drying the area around the skin; and
g. repeating steps (a) through (f) every 1, 2, 3, or 4 days until drainage from the injury stops;
wherein the injury involves bone exposure.

15. The method of claim 14, wherein after drainage stops the method further comprises the steps of:
a. removing the protective cover and occlusive dressing;
b. cleaning and drying the adjacent normal skin;
c. contacting the injury with a silver ion based topical pharmaceutical composition, wherein normal skin adjacent to the injury is not contacted with the silver ion based topical pharmaceutical composition;
d. re-covering the injury and adjacent normal skin with an occlusive dressing and protective cover; and
e. securing the protective cover over the injury.

16. The method of claim 15, wherein steps (a) through (e) are repeated every 1, 2, 3, or 4 days until the formation of the precursor of would be regenerated tissues.

17. The method of claim 16, wherein after the precursor has formed, steps (a) through (e) are repeated every 3 or 4 days until the injury is covered with dermis.

18. The method of claim 17, wherein after dermis has covered the injury, steps (a) through (e) are repeated weekly until epidermis is formed over the dermis.

19. The method of claim 18, wherein after epidermis is formed, the epidermis is contacted with a silver ion based topical pharmaceutical composition and covered with a protective cover, which are changed at least one time daily until treatment is complete.

20. The method of claim 11, wherein steps (a) through (e) are repeated every 1, 2, 3, or 4 days until drainage stops.

21. The method of claim 11, wherein the standard dressing is gauze or perforated occlusive dressing and gauze.

22. The method of claim 11, wherein the injury is a digit injury.

23. The method of claim 11, wherein the silver ion based topical pharmaceutical composition is a silver sulfadiazine topical pharmaceutical composition.

24. A kit for managing a multi-tissue injury comprising:
a. an amount of silver ion based topical pharmaceutical composition;
b. at least one standard dressing;
c. at least one protective cover; and
d. at least one occlusive dressing.

25. The kit of claim 21, wherein the standard dressing is gauze or perforated occlusive dressing.

26. The kit of claim 21, wherein the silver ion based topical pharmaceutical composition is a silver sulfadiazine topical pharmaceutical composition.

27. A method of treating a multi-tissue injury comprising of contacting the injury with a silver ion based topical pharmaceutical composition, the improvement comprising avoiding exposure of the injury to air and water, for a time that permits regeneration of damaged tissues.

28. The method of claim 24, wherein the injury is covered with a protective dressing.

29. The method of claim 24, wherein trauma to the injury is avoided during treatment.

30. The method of claim 27, wherein the injury is a digit injury.

31. The method of claim 27, wherein the silver ion based topical pharmaceutical composition is a silver sulfadiazine topical pharmaceutical composition.
